(19) United States
(12) Patent Application Publication
(10) Pub. No.: US 2022/0015713 A1
(43) Pub. Date: Jan. 20, 2022

(54) MACHINE LEARNING QUALITY ASSESSMENT OF PHYSIOLOGICAL SIGNALS

(71) Applicant: SENSORITY LTD., Ashdod (IL)

(72) Inventors: Maria KAMINSKY, Ashdod (IL); Bar MORDEHAY, Beit Arye (IL); Dmitry GOLDENBERG, Ashdod (IL)

(21) Appl. No.: 17/376,450

(22) Filed: Jul. 15, 2021

Related U.S. Application Data
(60) Provisional application No. 63/052,573, filed on Jul. 16, 2020.

(51) Int. Cl. A61B 5/00; A61B 5/024; A61B 5/352; G06N 20/00

(52) U.S. Cl. CPC A61B 5/7267; G06N 20/00; A61B 5/352; A61B 5/02416

(57) ABSTRACT

A method comprising receiving, as input, a plurality of PPG waveform signal segments; extracting, from each of the segments, a feature set representing the PPG waveform signal; at a training stage, training a machine learning model on a training set comprising: (i) the feature sets, and (ii) labels indicating a quality parameters associated with the PPG waveform signal in each of the PPG waveform signal segments; and at an inference stage, applying the trained machine learning model to at least one feature set extracted from at least one target PPG waveform signal segment, to determine a quality parameter of the at least one target PPG waveform signal.

### CROSS-REFERENCE TO RELATED APPLICATIONS

This application claims the benefit of priority of U.S. Patent Application No. 63/052,573, filed Jul. 16, 2020, which is hereby incorporated by reference in its entirety.

### FIELD OF THE INVENTION

The present invention relates generally to the field of machine learning. More specifically, the present invention relates to using machine learning for quality assessment of physiological signals.

### BACKGROUND OF THE INVENTION

Photoplethysmography (PPG) is a non-invasive optical technique to detect blood-volume variations in the microvascular bed of tissue. This is possible because blood has a higher absorption than bloodless skin, so an increase in blood-volume will decrease the intensity of the light reflected from the skin. PPG-based devices have been proven effective to monitor relevant physiological variables that include cardiac activity, respiration, and thermal regulation.

The change in volume caused by the pressure pulse is detected by illuminating the skin and then measuring the amount of light either transmitted or reflected. Because blood flow to the skin can be modulated by multiple other physiological systems, the PPG can also be used to monitor breathing, hypovolemia, and other circulatory conditions.

However, optically-based pulsatile signals are often corrupted with noise artifacts, which hampers accuracy of the detection and consequently leads to inaccurate heart rate estimation.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, in an embodiment, a system comprising at least one hardware processor; and a non-transitory computer-readable storage medium having stored thereon program code, the program code executable by the at least one hardware processor to: receive, as input, a plurality of signal waveform segments, each representing an optically-obtained physiological signal, extract, from each of the segments, a feature set representing the respective signal waveform in the segment, at a training stage, train a machine learning model on a training set comprising: (i) the feature sets, and (ii) labels indicating a quality parameters associated with the signal waveform in each of the respective segments, and at an inference stage, apply the trained machine learning model to at least one feature set extracted from at least one target signal waveform segment, to determine a quality parameter of the at least one target signal waveform.

There is also provided, in an embodiment, a method comprising receiving, as input, a plurality of signal waveforms segments, each representing an optically-obtained physiological signal; extracting, from each of the segments, a feature set representing the respective signal waveform in the segment; at a training stage, training a machine learning model on a training set comprising: (i) the feature sets, and (ii) labels indicating a quality parameters associated with the signal waveform in each of the respective segments; and at an inference stage, applying the trained machine learning model to at least one feature set extracted from at least one target signal waveform segment, to determine a quality parameter of the at least one target signal waveform.

There is further provided, in an embodiment, a computer program product comprising a non-transitory computer-readable storage medium having program instructions embodied therewith, the program code executable by at least one hardware processor to receive, as input, a plurality of signal waveform segments, each representing an optically-obtained physiological signal; extract, from each of the segments, a feature set representing the respective signal waveform in the segment; at a training stage, train a machine learning model on a training set comprising: (i) the feature sets, and (ii) labels indicating a quality parameters associated with the signal waveform in each of the respective segments; and at an inference stage, apply the trained machine learning model to at least one feature set extracted from at least one target signal waveform segment, to determine a quality parameter of the at least one target signal waveform.

In some embodiments, the feature sets are labeled with said labels.

In some embodiments, the optically-obtained physiological signal is a photoplethysmography (PPG) signal.

In some embodiments, each of said feature sets comprises at least one feature selected from the group consisting of: count of RR peaks, count of NN peaks, ratio of RR peaks to NN peaks, spectrum peak amplitude, spectrum area, RR variance, NN variance, outlier distance, and correlation curve fitting.

In some embodiments, each of said signal waveform segments is extracted from a video sequence.

In some embodiments, the program instructions are further executable to apply, and the method further comprises applying, said trained machine learning model to a continuous sequence of signal waveform segments, to predict a quality parameter of said signal waveform at each point in time in said continuous sequence.

In some embodiments, each pair of consecutive said segments in said sequence are overlapping, at least in part.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is a flowchart of the functional steps in a process for training a machine learning model to predict signal quality of a physiological signal, according to an embodiment of the present invention;

FIGS. 2A-2B are flowcharts of the functional steps in an algorithm for continuous real-time PPG signal quality assessment, using a trained machine learning model of the present disclosure, in accordance with some embodiments;

FIGS. 3A-3G show boxplots of extracted features, according to an embodiment of the present invention; and

FIGS. 4A-4C and 5A-5C show experimental results, according to an embodiment of the present invention.

### DETAILED DESCRIPTION

Disclosed herein are a system, method, and computer program product for real-time machine-learning based evaluation and validation of physiological signal quality.

Signal quality or signal-to-noise ratio requires consideration in almost all signal measurements. This is especially true in physiological measurements, where the signals tend to be weak and prone to measurement artefacts, and where the noise component is often difficult to control.

Examples and embodiments detailed herein will discuss extensively aspects of the present invention with respect to optically-obtained physiological signals, such as photoplethysmography (PPG) signals. However, the present disclosure may apply to additional and/or other types of optically-obtained physiological signals, including, but not limited to, bodily temperature, skin surface temperature, respiratory rate, heart rate, muscle tension, and the like.

A photoplethysmogram (PPG) is an optically-obtained plethysmogram that can be used to detect blood volume changes in the microvascular bed of tissue. Remote photoplethysmography allows to determine physiological processes, such as blood flow, without skin contact. This is achieved by using, e.g., a video stream of a suitable skin region of a subject (e.g., face) to analyze subtle momentary changes in skin color, which are not detectable to the human eye. Such camera-based measurement of blood oxygen levels provides a contactless alternative to conventional photoplethysmography.

However, remote PPG may be susceptible to noise and artifacts in the signal, as a result of, e.g., motion of the subject, changes and inconsistencies in ambient lighting, and related noise and artifact issues.

Accordingly, in some embodiments, the present disclosure provides for evaluating quality of an optically-obtained physiological signal (e.g., PPG), to detect and identify portions of signals with noise and artifacts that may adversely affect the ability to extract useful physiological information from the signal.

For example, as known in the art, an optically-obtained physiological signal (e.g., PPG) may be used to determine one or more subsequent physiological, diagnostic parameter values, including for example heart-rate, blood flow, blood pressure, blood oxygenation levels, ankle-brachial index (ABI), toe-brachial index (TBI), R-peak amplitude, and the like. The PPG signal and/or the one or more physiological, diagnostic parameter values may subsequently be used by a physician, to produce a diagnosis of a subject's (e.g., a patient's) medical condition. Embodiments of the invention may enable diagnosis of the subject based on a quality parameter of said optically-obtained physiological signal (e.g., PPG). For example, embodiments of the invention may produce an indication of a quality parameter (e.g., Signal to Noise (SNR)) of said at least one optically-obtained physiological signal and/or physiological, diagnostic parameter values, to indicate whether said diagnosis is reliable or not.

Additionally, or alternatively, embodiments of the invention may include, or may be associated with an automated (e.g., machine-learning based) assistive diagnostic system. Embodiments of the invention may provide to the assistive diagnostic system, an indication of a quality parameter (e.g., SNR) of the optically-obtained physiological signal (e.g., PPG) physiological and/or said diagnostic parameter values. According to some embodiments, the assistive diagnostic system may subsequently produce a prediction (e.g., diagnosis, prognosis, etc.) of a subject's medical condition based on the indication of a quality.

For example, if the optically-obtained physiological signal is indicated as noisy, then the assistive diagnostic system may not produce a prediction of the subject's medical condition based on the noisy signal. In a complementary manner, if the optically-obtained physiological signal is indicated as clean, the assistive diagnostic system may produce a prediction of the subject's medical condition based on the clean signal.

In some embodiments, a machine learning model is trained on a dataset comprising features obtained from a plurality of optically-obtained physiological signal segments, wherein the dataset is annotated with labels representing signal quality. In some embodiments, the extracted features are in the time and frequency domains. In some embodiments, a least five features are extracted from each dataset sample. In some embodiments, the machine learning model is a Gaussian SVM. In some embodiments a machine learning model of the present disclosure has a total prediction accuracy of 92%.

In some embodiments, a trained machine learning model of the present disclosure may provide for real-time prediction and/or classification of optically-obtained physiological signal quality, based on an evaluation algorithm which continuously evaluates, in real-time, consecutive overlapping segments of a signal. In some embodiments, a real-time assessment algorithm of the present disclosure generates a progression representation that finds at each step the quality of the next signal segment.

In some embodiments, a real-time assessment algorithm of the present disclosure outputs a signal timeline index associated an acquired signal, which identifies signal segments associated with artifactual and/or noisy and/or inadequate quality signal.

### BACKGROUND

Optically-based physiological signals, e.g., PPG, may be acquired from a video sequence of a subject. Typically, a suitable region of interest (ROI) of the subject's skin has to be detected and tracked through the video sequence, where the efficiency of ROI selection eventually determines the quality and validity of the extracted signal. Facial regions are a good candidate since they are most often accessible and because they represent a relatively high cutaneous perfusion.

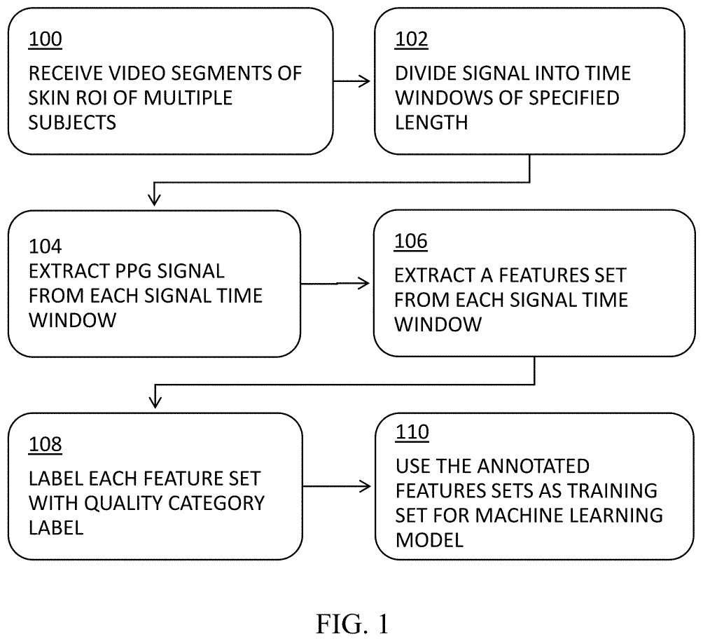

FIG. 1 is a flowchart of the functional steps in a process for training a machine learning model to predict and/or classify signal quality of a physiological signal extracted from an optically-based physiological signal acquisition methodology, according to an embodiment of the present invention.

### Training Set Construction

In some embodiments, a machine learning model may be trained to predict and/or classify signal quality of a physiological signal extracted from an optically-based physiological signal acquisition methodology, e.g., PPG.

With continuing reference to FIG. 1, at step 100, in some embodiments, a training dataset for training a machine learning model of the present disclosure includes receiving a plurality of signal segments obtained from one or more video sequences. In some embodiments, the video sequences may be obtained from one or more, e.g., a plurality of different human subjects. In some embodiments, the video segments depict suitable skin ROIs of the subjects, e.g., facial skin area.

In some embodiments, the video data may be acquired using a number of acquisition modalities, e.g. RGB, monochrome imaging, near infrared (NIR), short-wave infrared (SWIR), infrared (IR), ultraviolet (UV), multi spectral, hyperspectral, and/or any other and/or similar imaging techniques. In some embodiments, the video acquisition modalities may represent varying frame rates (e.g., between 15-50 frames per second (fps) or higher rates), and/or different light conditions, ambient environments, and subject movements.

In some embodiments, at step 102, a training set according to the present disclosure may comprise video data obtained from one or more video sequences. In some embodiments, the video sequences may be divided into time window segments comprising, e.g., 10 seconds each, or between 5 and 20 seconds each. In some embodiments, the time windows are independent and not overlapping with one another.

In some embodiments, at step 104, a physiological signal, e.g., a PPG signal, may be calculated from each of the time windows.

### Feature Extraction

In some embodiments, at step 106, a feature extraction step is performed, wherein characteristics that are unique to the particular physiological signal are extracted from each signal segment. In some embodiments, these features reflect the fact that PPG is a sinusoidal waveform-like signal with a permanent time period without artifacts or any random noise. For example, for each signal time window there are extracted, e.g., the following features in a non-limiting embodiment:

### NN Over RR Peaks

This feature is based on the count of RR peaks and NN peaks in the signal. This feature is based on the fact that in PPG signal, like any sinusoidal waveform, there are clear peaks for each interval. The RR-interval refers to the time between two R-peak of a traditional ECG heart-beat waveform, while NN-intervals refer to the intervals between normal R-peaks. The division of these two parameters may involve the number of normal peaks relatively to all peaks. As a result, for a signal of a good quality, it is expected to receive a number that is closer to 1, while for a noisy signal it is expected to receive a number that is closer to 0.

$$\frac{NN}{RR} = \frac{counter of NN}{counter of RR} \qquad ( 1 )$$

### Spectrum Peak Amplitude

This feature comprises calculating, for each signal time window, the spectrum by Fourier Transforms:

spectrumWindow=|FFT{PPGₛignal_{w}indow}| (2)

Then, for each signal time window channel, find the two maximum magnitudes of the spectumWindow. Divide the two magnitudes:

$$spectrumPeakAmplitude = \frac{smaller magnitude}{larger magnitude} \qquad ( 3 )$$

This feature helps to find if there is a noise in the signal's spectrum. The main idea is that a clear signal will contain one clear peak at the spectrum that depicts the main frequency of the PPG signal. However, a PPG signal that contains a lot of noise includes more than one clear peak. Therefore, when the PPG spectrum contains noise, this feature acquires a value that is very close to 1, while for low noise signal, it is expected to receive a value that is closer to 0.

### Spectrum Area

This feature comprises, for each signal time window, first calculating the spectrum by Fourier Transforms (Eq. 2), then calculate the area under the curve of this signal time window spectrum:

spectumArea=∫(spectumWindow)∂f (4)

The main idea of this feature is that a spectrum of a clean signal contains a clear peak and looks like a delta function, while a spectrum of a noisy signal contains a lot of peaks so the spectrum's area will be higher.

### RR Variance

This feature comprises calculating, for each signal time window the variance of the RR interval. First, the time intervals between the peaks of the PPG signal time window in the time domain is extracted. Then, the variance is calculated by:

$$\begin{matrix}Mean\_Intervals = \frac{1}{N} {\sum }_{i = 1}^{N} RR\_Interval [ i ] , \\ N = number of RR\_Intervals & ( 5 ) \\ Variance\_RR = \frac{1}{N} {\sum }_{i = 1}^{N} {( Mean\_Intervals - RR\_ Interval [ i ] )}^{2} & ( 6 )\end{matrix}$$

The main idea of this feature is that when the PPG signal's time period is constant, the variance will be very low and close to 0, while in noisy PPG signal, it is very hard to find the time period and the variance will be very high.

### NN Variance

This feature This feature comprises calculating, for each signal time window the variance of the RR interval. First, the time intervals between normal R-peaks are extracted. Then, the variance is calculated by:

$$\begin{matrix}Mean\_Intervals = \frac{1}{N} {\sum }_{i = 1}^{N} NN\_Interval [ i ] , \\ N = number of NN\_Intervals & ( 7 ) \\ Varianc\_NN = \frac{1}{N} {\sum }_{i = 1}^{N} {( Mean\_Intervals - NN\_ Interval [ i ] )}^{2} & ( 8 )\end{matrix}$$

The main idea and the calculation of this feature are the same as with RR variance. The difference in this feature is that is this case the calculation is done for the NN intervals (only the normal peaks that were picked from the R peaks).

### Outliers Distance

This feature reflects the constancy value of the distance between the RR peaks remain. In good quality PPG signal, the shape should be like a sinusoidal wave which lead to constant peaks and constant time period. However, in noisy PPG signal, the duration from peak to the peak may change. The calculation of this feature:

$$outlier\_distance = \frac{1}{N} {\sum }_{i = 1}^{N} ( Median\_RR \_interval [ i ] ) \qquad ( 9 )$$

The median is the RR interval median of the PPG signal time window in the time domain, and N is the number of RR intervals in this signal time window.

### Correlation Curve Fitting

This feature comprises producing, for each signal time window, a sinusoidal curve fitting from an assumption that the signal time window signal should resemble a sinusoidal function. A correlation is then calculated between the curve fitting and the actual PPG signal time window. A high correlation (closer to 1) depicts a good PPG signal, while a lower correlation (closer to 0) depicts a noisy PPG signal.

### Training Set Annotation

In some embodiments, at step 108, each calculated signal time window may be rated and annotated with respect to signal quality reflected therein, e.g., into two or more quality categories. For example, the categories may comprise “good quality,” “medium quality,” and “noisy.” In other embodiment, fewer or more quality categories may be used. For example, only two categories may be used (e.g., “good,” and “noisy”).

In some embodiments, the signal time window annotations are performed manually by specialists, e.g., based on majority annotation among 3 specialists, or using any other annotation scheme.

In some embodiments, a training set of the present disclosure may comprise, e.g., an identical and/or similar number of signal segment sin each quality category.

### Training a Machine Learning Model

In some embodiments, at step 110, the present disclosure provides for training a machine learning model on a training dataset comprising a plurality of annotated feature sets associated with a plurality of time window segments acquired as detailed herein above. In some embodiments, the machine learning model may be any one of, e.g., SVM, KNN, Random Forest, etc.

In some embodiments, a trained machine learning model of the present disclosure may be configured for inferring on a target PPG signal segment to predict and/or classify a quality rating or quality parameter of the target segment. The quality parameter may be, or may represent for example a signal-to-noise (SNR) ratio.

### Real Time PPG Signal Quality Assessment

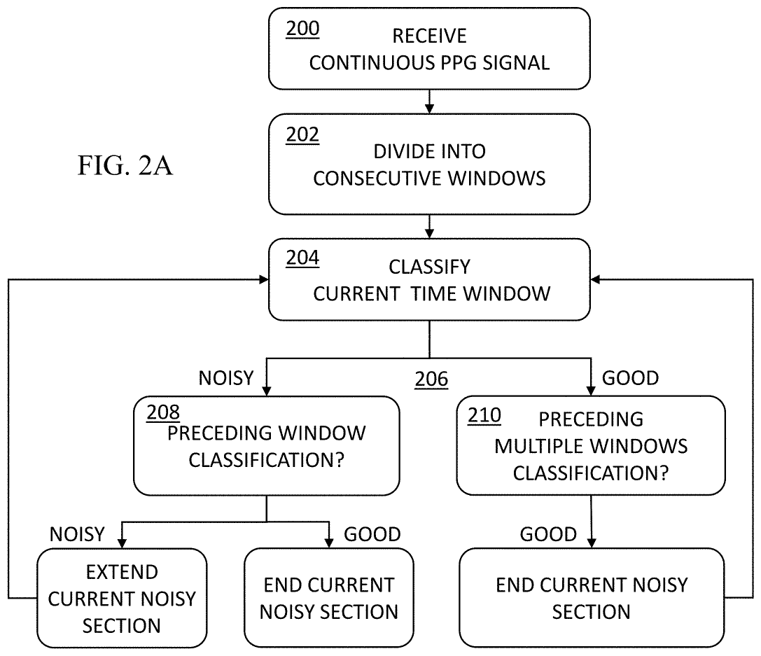

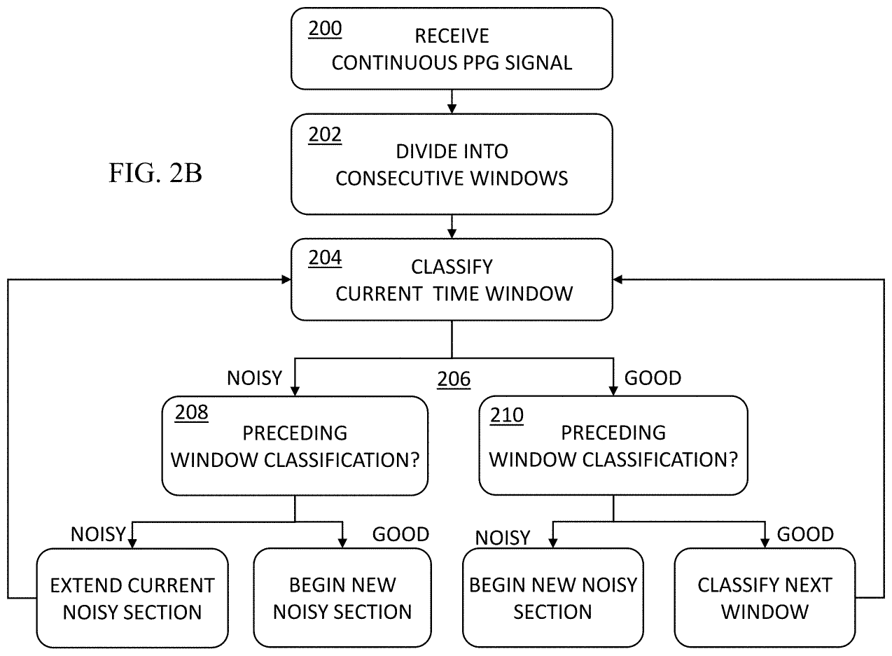

FIGS. 2A-2B are flowcharts of the functional steps in an algorithm for continuous real-time PPG signal quality assessment, using a trained machine learning model of the present disclosure, in accordance with some embodiments. In some embodiments, a trained machine learning model of the present disclosure may be used to predict and/or classify the quality of a continuous PPG signal at each point in time, to detect signal portions comprising noise and/or artifacts.

In some embodiments, the output of the present algorithm is a timetable indicating start and end times of segments of the continuous signal reflecting noisy and/or artifactual signal.

In some embodiments, at step 200, a continuously-acquired PPG signal may be received, based, e.g., on a continuous video stream of a target subject. In some embodiments, the video stream may be acquired in real time. In some embodiments, the video stream may depict a suitable skin ROI of the target subject, e.g., facial skin area. In some embodiments, the video data may be acquired using a number of acquisition modalities, e.g. RGB, monochrome imaging, near infrared (NIR), sort-wave infrared (SWIR), infrared (IR), ultraviolet (UV), multi spectral, hyperspectral, and/or any other and/or similar imaging techniques. In some embodiments, the video acquisition modalities may represent varying frame rates (e.g., between 15-50 fps, or higher rates), and/or different light conditions, ambient environments, and subject movements.

In some embodiments, at step 202, the continuous PPG signal may be divided into consecutive overlapping signal time windows, e.g., overlapping window of 9 seconds (increase of 1 second).

In some embodiments, and step 204, a trained machine learning model of the present disclosure may be applied in real time to each consecutive signal time window, to generate a prediction and/or classification of each signal time window into one or more quality categories, e.g., “good quality” and “noisy.”

In some embodiments, at step 206-210 a signal timeline indexing process may take place, wherein the present algorithm generates a continuous indexing of the input signal, which divides the signal into consecutive segments based on their signal classification. In some embodiments, the process indexes the timeline with timestamps, based on start/stop times of sections reflecting signal classification (e.g., good quality signals and noisy signals).

With reference to FIG. 2A, in some embodiments, the process of steps 206-210 indicates start/stop or beginning/end timestamps with respect to the continuous signal, which timestamps mark segments of the continuous signals as reflecting either ‘good’ or ‘noisy’ signal quality. In some embodiments, start/stop timestamps are determined based on a classification of a current signal time window. In some embodiments, start/stop timestamps determination takes into account a classification of one or more preceding signal time windows, e.g., 10 signal time windows.

In some embodiments, the process determines:

a classification of a current signal time window, and

classification of one or more preceding signal time windows, and

updates timestamps (e.g., start timestamp and stop timestamp) associated with one or more ‘noisy’ sections on the signal timeline.

For example, the process of steps 206-210 may comprise:

Step 206: Check classification of current signal time window—

If classified as ‘noisy:

Step 208: Check classification of 1 previous signal time window—

If classified as ‘good’: The start time of the current window is timestamped as a new start time of a new ‘noisy’ section.

If classified as ‘noisy: The end time of the current window is timestamped as an end time of a preceding ‘noisy’ section.

If classified as ‘good’:

Step 210: Check classification of 10 previous signal time windows—

If immediately preceding 5 signal time windows were classified as ‘good,’ and at least three signal time windows were classified as ‘noisy’ in the 5 signal time windows before that: The start time of the current window is timestamped as an end time of a preceding ‘noisy’ section.

With reference to FIG. 2B, in some embodiments, with respect only to the first few (e.g., 10) signal time windows, the process may comprise:

Step 206: Check classification of current signal time window—

If classified as ‘noisy’:

Step 208: Check classification of 1 previous signal time window—

If classified as ‘good’: The start and end times of the current window are timestamped as the start and end times of a new ‘noisy’ section.

If classified as ‘noisy’: The end time of the current window is timestamped as an end of a preceding ‘noisy’ section.

If classified as ‘good’:

Step 210: Check classification of 1 previous signal time window—

If classified as ‘good’: Continue to classify the next signal time window.

If classified as ‘noisy’: The start time of the current window is timestamped as an end time of a preceding ‘noisy’ section.

### Experimental Results

### Feature Predictive Power

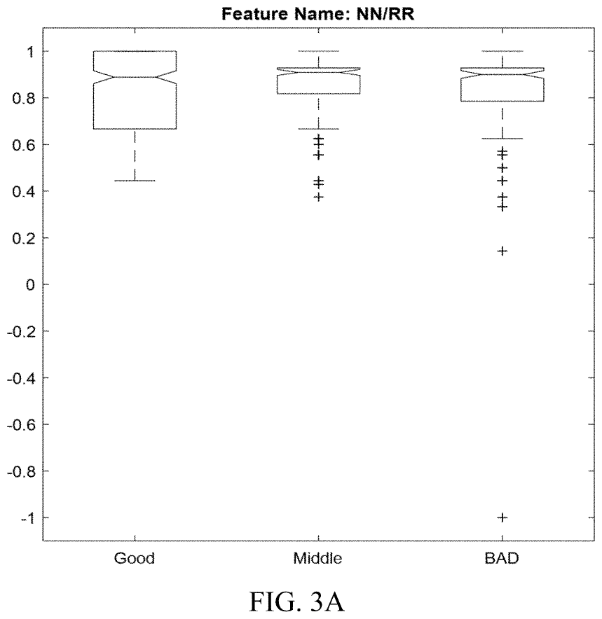

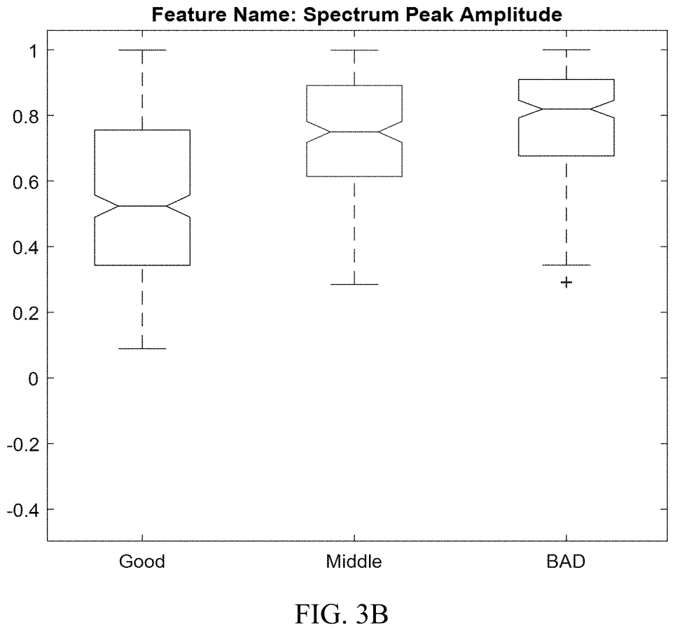

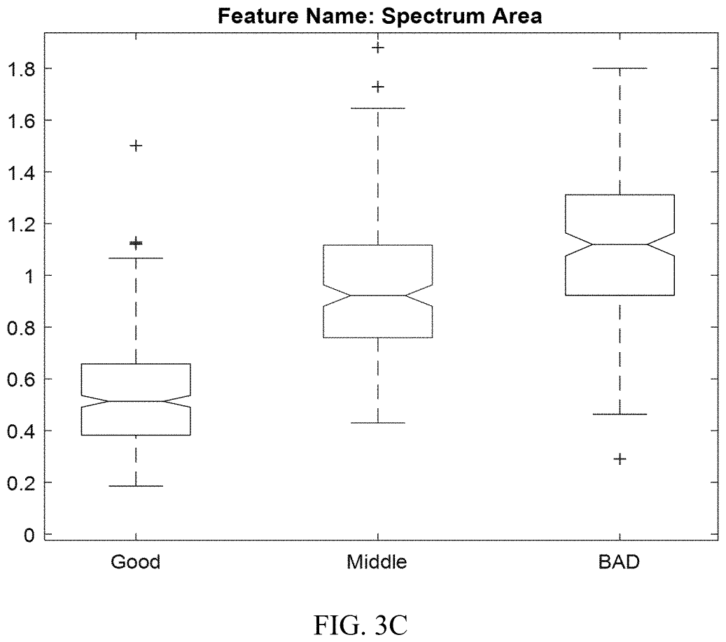

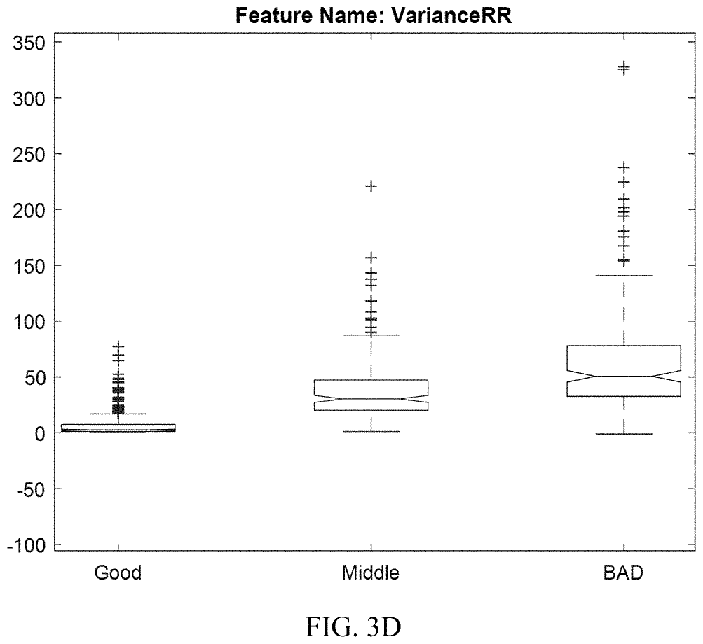

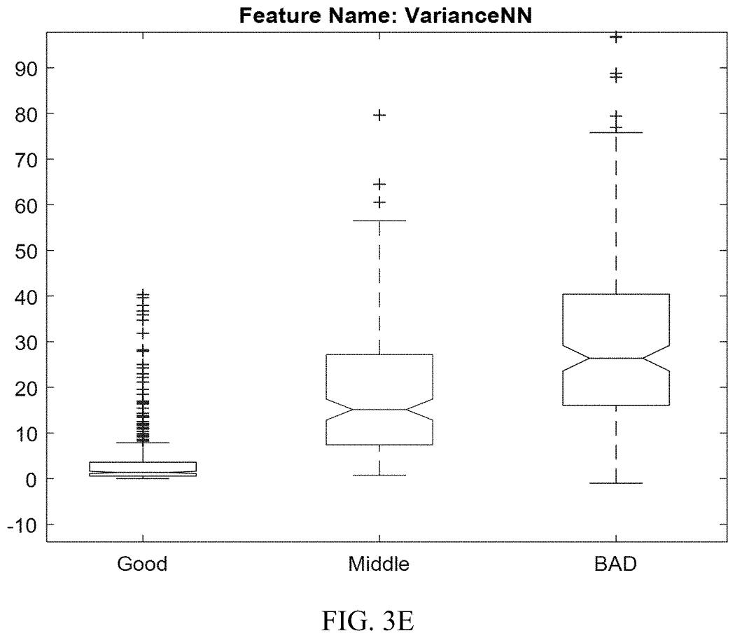

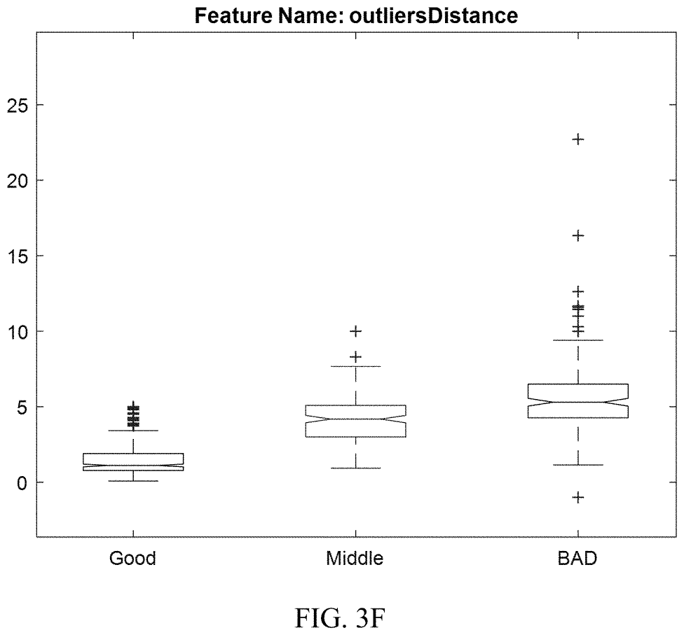

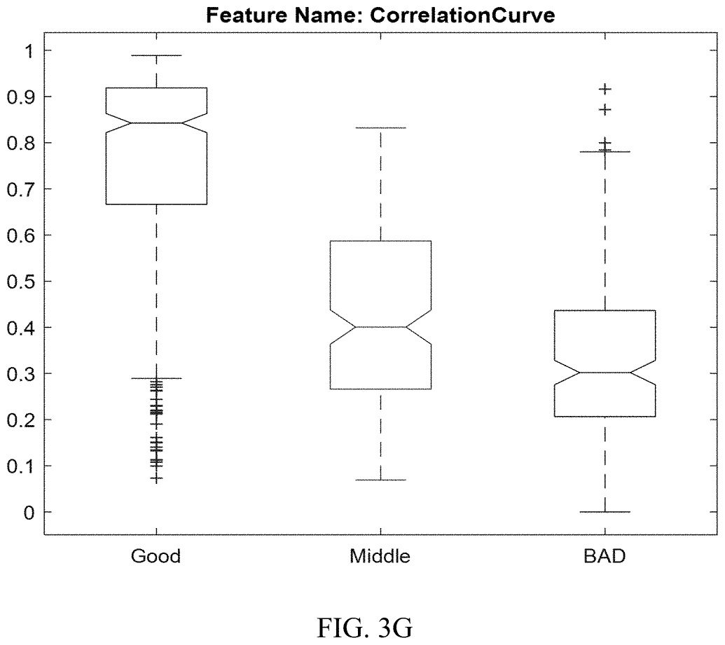

FIGS. 3A-3G illustrate features extracted, according to some embodiments. As can be seen, almost all the features exhibit good predictive power with respect to PPG signal quality, and specifically, between the “good” group and the other two groups (“middle” and “noisy” together).

To explore the predictive power of the various features, a t-test was performed. The results show that the features spectrum peak amplitude, spectrum area, RR variance, NN variance, outlier distance, and correlation curve fitting (FIGS. 3B-3G, respectively) reflect significant separation (P<<0.05). The feature represented in FIG. 3A, NN over RR Peaks, shows lower significance (p=0.2). RR variance and NN variance (FIGS. 3D-3E, respectively) represent similar concepts, however, NN variance offers better separation, and therefore it was retained over RR variance. Accordingly, of the 7 features depicted discussed herein, only the following were used in the experimental setup:

Spectrum peak amplitude,

Spectrum area,

NN variance,

Outlier distance, and

Correlation curve fitting.

### Machine Learning Model Validation

Various machine learning models were explored using MATLAB classification learners. The models were trained on a training set constructed as discussed above, and validated on a validation set. The results are displayed in table 1 below.

TABLE 1
machine learning classification results for different models

| | True positive | True positive | Total |
|---|---|---|---|
| Model type | “good” | “noisy” | accuracy |
| SVM-Gaussian | 89% | 94% | 91.5% |
| Ensemble-Bagged | 91% | 97% | 94.2% |
| Trees | | | |
| KNN-weighted | 90% | 98% | 93.8% |

As can be seen, the best results of the training set prediction were for the Ensemble model, however, when the results were explored on a validation set, all models offered similar predictive performance. The precision percentages of the SVM model with respect to the validation set were similar to the training set, which means that this model was not overfitted.

The Gaussian SVM model was implemented in C++ programming language, with parameter Gamma: 0.56. The prediction performance results of this model are set forth in table 2.

TABLE 2
Gaussian SVM prediction results

| | True positive- | True positive- | Total |
|---|---|---|---|
| Model type | “good” | “noisy” | accuracy |
| SVM-Gaussian | 91.7% | 92.2% | 92% |

As can be seen, the model results of C++ programming language are the same as the results of MATLAB program.

### Real Time Algorithm Validation

Upon testing, the optimal division of the continuous signal into overlapping signal time windows comprises time windows window of 10 seconds with a 9 second overlap (i.e., increase of 1 second per window) generate the most accurate results.

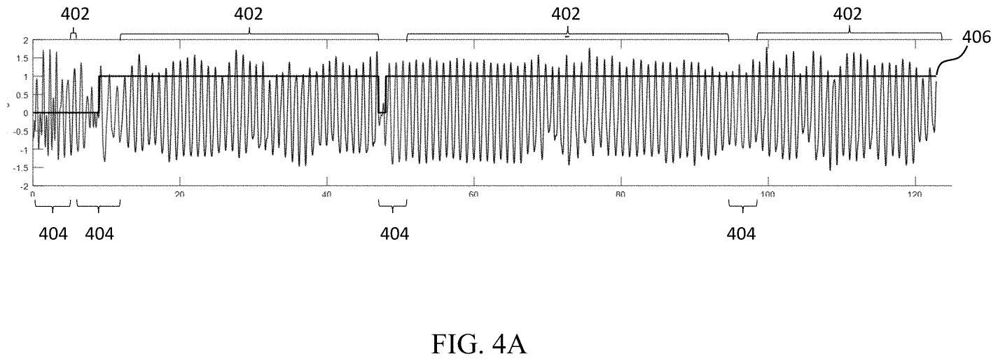

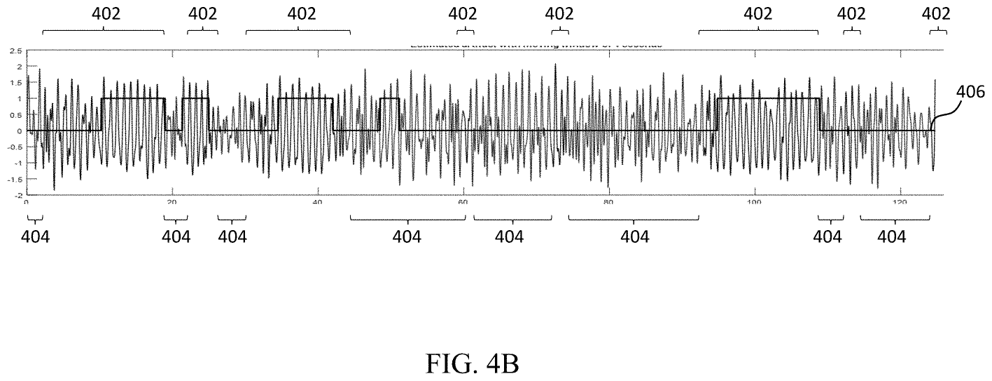

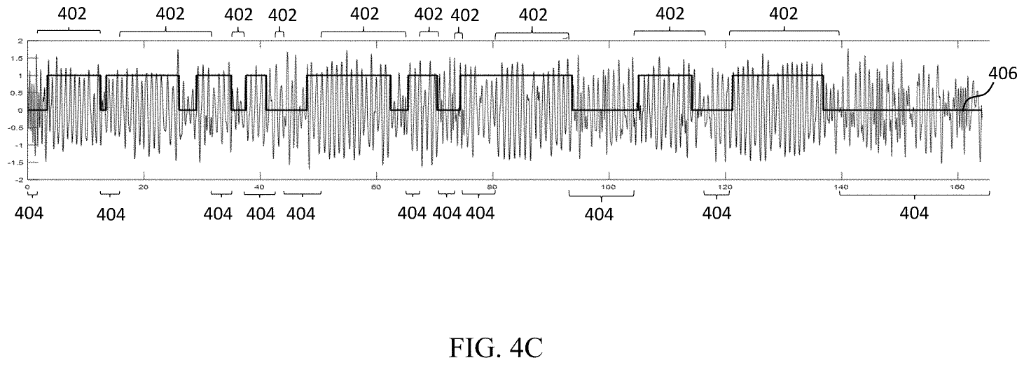

FIGS. 4A-4C show an output timeline of the present algorithm. The signals sections 404 marks the predicted artifacts and the signals sections 402 mark the predictions of good PPG signals. The step function 406 marks the labels of the PPG signal, where 0 indicates artifactual/noisy signal, and 1 indicates ‘good’ PPG signal. As can be seen, a majority of the artifactual sections were predicted correctly.

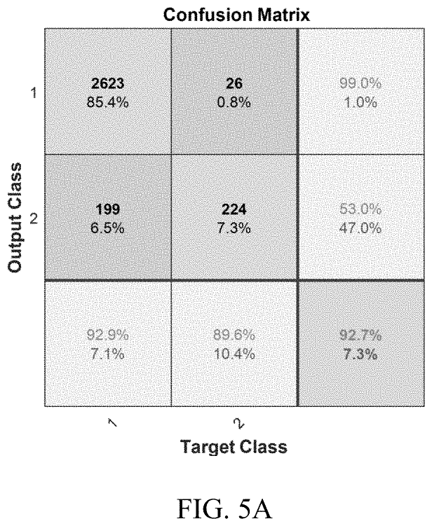

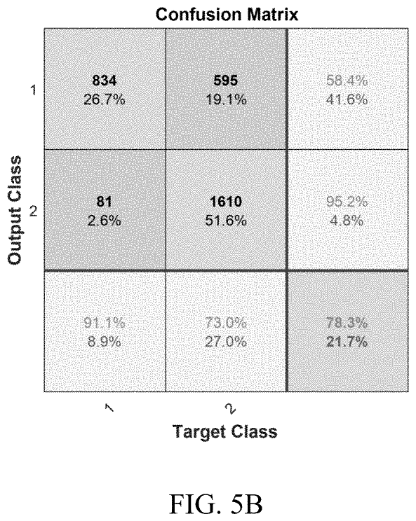

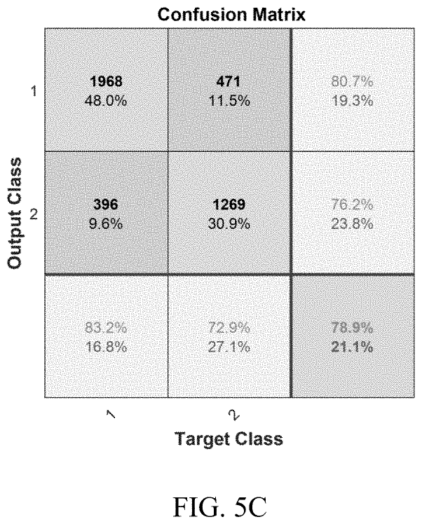

FIGS. 5A-5C are confusion matrices with respect to the signals shown in FIGS. 4A-4C, respectively. As can be seen, the classification of the ‘good’ signal sections (label “1”) performed well in all 3 signals (92.9%, 91.1%, 83.2%). The classification of the artifactual signal sections (label 2) performed in most cases (89.6%, 73%, 72.9%). It can be seen in FIGS. 4A-4C that misclassifications of the artifactual sections may be attributed to incorrect prediction of the start or the end time of the artifactual sections.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

What is claimed is:

1. A system comprising: at least one hardware processor and a non-transitory computer-readable storage medium having stored thereon program instructions, the program instructions executable by the at least one hardware processor to:
receive, as input, at least one signal waveform segment representing an optically-obtained physiological signal;
extract, from said at least one waveform segment, at least one respective feature set; and
apply a trained machine learning (ML) model to the at least one feature set to determine a quality parameter of said at least one signal waveform.

2. The system of claim 1, wherein the ML model is trained on a training set comprising: (i) a plurality of feature sets, extracted from a respective plurality of waveform segments, and (ii) labels indicating at least one quality parameter associated with said signal waveforms.

3. The system of claim 1, wherein said optically-obtained physiological signal is a photoplethysmography (PPG) signal.

4. The system of claim 1, wherein each of said feature sets comprises at least one feature selected from the group consisting of: count of RR peaks, count of NN peaks, ratio of RR peaks to NN peaks, spectrum peak amplitude, spectrum area, RR variance, NN variance, outlier distance, and correlation curve fitting.

5. The system of claim 1, wherein said quality parameter indicates whether said signal waveform is a noisy signal waveform.

6. The system of claim 1, wherein said at least one signal waveform segment comprises a sequence of signal waveform segments representing a continuous signal waveform, and wherein said program instructions are further executable to apply said trained machine learning model to each of said segments in said sequence, to generate said determining with respect to each of said segments in said sequence.

7. The system of claim 6, wherein said program instructions are further executable to output an index indicating beginning and end points of sections in said continuous signal waveform comprising a noisy signal waveform, based, at least in part, on said determining.

8. A method comprising:
receiving, as input, a at least one signal waveforms segment representing an optically-obtained physiological signal;
extracting, from said at least one waveform segment, at least one respective feature set; and
applying a trained ML model to the at least one feature set to determine a quality parameter of said at least one signal waveform.

9. The method of claim 8, wherein the ML model is trained on a training set comprising: (i) a plurality of feature sets, extracted from a respective plurality of waveform segments, and (ii) labels indicating at least one quality parameter associated with said signal waveforms.

10. The method of claim 8, wherein said optically-obtained physiological signal is a photoplethysmography (PPG) signal.

11. The method of claim 8, wherein each of said feature sets comprises at least one feature selected from the group consisting of: count of RR peaks, count of NN peaks, ratio of RR peaks to NN peaks, spectrum peak amplitude, spectrum area, RR variance, NN variance, outlier distance, and correlation curve fitting.

12. The method of claim 8, wherein said quality parameter indicates whether said signal waveform is a noisy signal waveform.

13. The method of claim 8, wherein said at least one signal waveform segment comprises a sequence of signal waveform segments representing a continuous signal waveform, further comprising applying said trained machine learning model to each of said segments in said sequence, to generate said determining with respect to each of said segments in said sequence.

14. The method of claim 13, further comprising outputting an index indicating beginning and end points of sections in said continuous signal waveform comprising a noisy signal waveform, based, at least in part, on said determining.

15. A computer program product comprising a non-transitory computer-readable storage medium having program instructions embodied therewith, the program instructions executable by at least one hardware processor to:
receive, as input, a plurality of signal waveform segments, each representing an optically-obtained physiological signal;
extract, from each of said segments, a feature set representing said respective signal waveform in said segment;
at a training stage, train a machine learning model on a training set comprising:
(i) said feature sets, and
(ii) labels indicating a quality parameters associated with said signal waveform in each of said respective segments; and
at an inference stage, apply said trained machine learning model to at least one feature set extracted from at least one target signal waveform segment, to determine a quality parameter of said at least one target signal waveform.

16. The computer program product of claim 15, wherein said optically-obtained physiological signal is a photoplethysmography (PPG) signal.

17. The computer program product of claim 15, wherein each of said feature sets comprises at least one feature selected from the group consisting of: count of RR peaks, count of NN peaks, ratio of RR peaks to NN peaks, spectrum peak amplitude, spectrum area, RR variance, NN variance, outlier distance, and correlation curve fitting.

18. The computer program product of claim 15, wherein said quality parameter indicates whether said signal waveform is a noisy signal waveform.

19. The computer program product of claim 15, wherein said at least one target signal waveform segment comprises a sequence of signal waveform segments representing a continuous signal waveform, and wherein said program instructions are further executable to apply said trained machine learning model to each of said segments in said sequence, to generate said determining with respect to each of said segments in said sequence.

20. The computer program product of claim 19, wherein said program instructions are further executable to output an index indicating beginning and end points of sections in said continuous signal waveform comprising a noisy signal waveform, based, at least in part, on said determining.
